(19) United States
(12) Patent Application Publication
(10) Pub. No.: US 2003/0091627 A1
(43) Pub. Date: May 15, 2003

(54) Rate-controlled delivery of macrolides

(76) Inventor: Vinay Sharma, Long Valley, NJ (US)

Correspondence Address: Louis E. Marn, 9 Edgewater Drive, Palm Coast, FL, 32164, US

(21) Appl. No.: 10/283,902

(22) Filed: Oct. 30, 2002

Related U.S. Application Data
(60) Provisional application No. 60/336,478, filed on Oct. 31, 2001.

(51) Int. Cl. A61K 31/7048; A61K 31/724; A61K 9/20

(52) U.S. Cl. 424/465; 514/28; 514/58

(57) ABSTRACT

There is disclosed the formulation of a poorly soluble macrolide antibiotic, such as clarithromycin together with β-cyclodextrin, and optionally a dicarboxylic acid wherein the particles of the formulation are prepared using microfluidization techniques in a particle size in the range of from 5 to 15 microns.

### BACKGROUND OF THE INVENTION

1. Field of the Invention

The present invention relates to a drug delivery system, and more particularly to a rate-controlled delivery for once daily dosing of a macrolide.

2. Related Application

This application claims the benefit of U.S. Provisional application No. 336,478, filed Oct. 31, 2001.

3. Description of the Prior Art

Clarithromycin is a semi-synthetic antimicrobial 14-member macrolide exhibiting a broad in-vitro antibacterial spectrum. Structurally, Clarithromycin differs from erythromycin only in the substitution of an o-metyl group for the hydroxyl group at position six of the lactone, with increased tissue or cellular penetration, attributed to formation of a microbiologically-active metabolite, 13®-hydroxy-clarithromycin. It has a more favorable pharmacokinetic profile, than erythromycin, which allows twice-daily administration and a possible increase in compliance. To improve the spectrum of activity and decrease the disadvantages of erythromycin, a new generation of macrolide antibiotics has been developed.

These include azithromycin, clarithromycin, roxithromycin, dirithromycin, micomycin and rokitamycin.

Clarithomycin was discovered and patented by Taisho Pharmaceutical Co. Ltd. Japan (U.S. Pat. No. 4,331,803) and is being marketed by Abbott Laboratories. Clarithromycin IR tablets were first approved by US FDA on Oct. 31, 1991, and are currently administered at doses of 250 and 500 mg to permit greater patient convenience and compliance. An ER tablet for once daily dosage has been developed.

In U.S. Pat. No. 5,705,190, there is described controlled release, oral, solid, pharmaceutical composition for a reduced daily dosage regimen where the therapeutic ingredient is a poorly soluble basic drug. The formulation comprises the use of a water-soluble alginate salt, a complex salt of alginic acid and an organic carboxylic mixture with the therapeutic drug. A particular embodiment comprises once daily dosage form of clarithromycin is disclosed. Prior art describes the applications of multicomponent complexes of sparingly water-soluble amino-type drug, a cyclodextrin and a hydroxycarboxylic acid. Simultaneous complexation and salt formation with such acids significantly increase the solubilizing power allowing a reduced amount of cyclodextrin necessary for making the targeted formulation.

Clarithromycin had a molecular weight of 747.96, pKa (not easily available from literature), and is stable at an acidic pH. Solubility is 1 in 1000 parts of water. One of the most important application of cyclodextrins (CDs) is the possibility of increase in the aqueous solubility of sparingly soluble drugs, especially in alternative to other techniques, such as the use of co-solvents or surfactants for microemulsion formation. However, natural CDs are generally not very soluble in water because of the relatively strong binding of the molecules in the crystal state (i.e. relatively high crystal lattice energy). In particular, β-cyclodextrin (BCD), the most widely used member of the family by virtue of its cavity size, availability and low cost exhibiting limited solubility (1.85% at 25° C.) often hinders it successful application as a solubilizing agent. The solubility of clarithromycin presents a problem in effective usage.

### OBJECTS OF THE INVENTION

An object of the present invention is to provide an effective drug delivery system for macrolides obviating solubility problems.

Another object of the present invention is to provide an effective once daily delivery system for macrolides.

Still another object of the present invention is to provide an effective once daily delivery system for clarithromycin.

### SUMMARY OF THE INVENTION

These and other objects of the present invention is achieved by a formulation of a poorly soluble macrolide antibiotics, particularly clarithromycin together with B-cyclodextrin, and optionally a dicarboxylic acid wherein the particles of the formulation is prepared using microfluidizing techniques in a particle size in the range of 5 to 15 microns.

### BRIEF DESCRIPTION OF THE DRAWINGS

A better understanding of the present invention will be obtained by consideration of the following detailed description thereof when taken with the accompanying drawings wherein:

FIG. 1. is a plot of plasma concentration-time computed for one (1) mg. of clarithromycin;

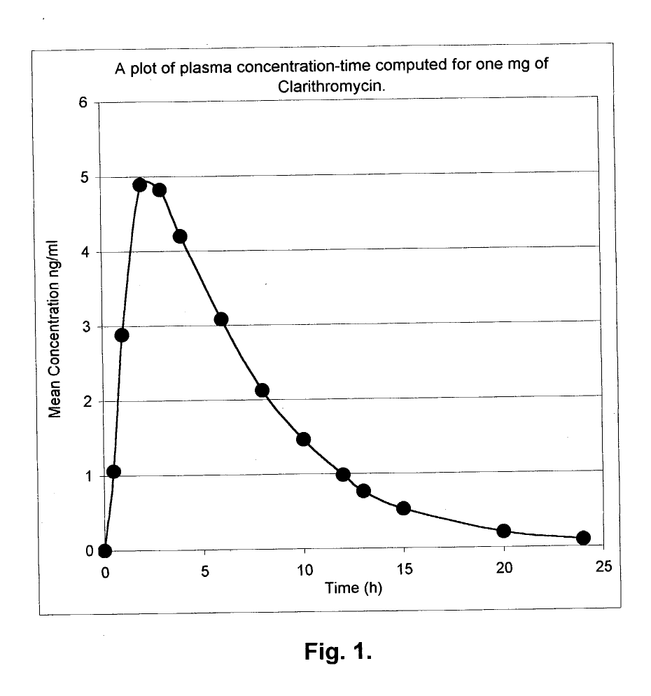

FIG. 2. is a plot of plasma concentration-time for Biaxin®-XL, 500 mg.;

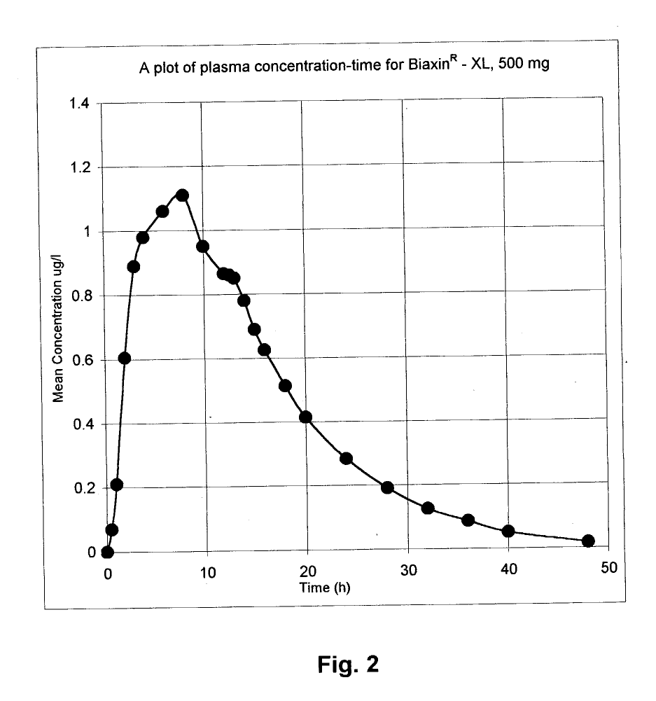

FIG. 3 is a plot comparing in-vivo outputs in terms of cumulative amounts (mg) for Biaxin®-XL, 500 mg and 1000 mg.: and

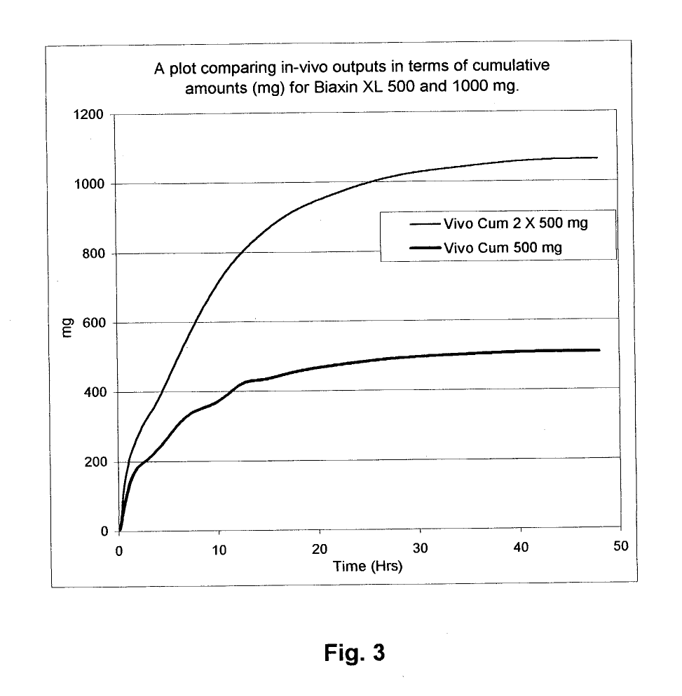

FIG. 4 is a plot of in-vitro to in-vivo correlation based on fraction response times for Biaxin®-XL, 500 mg.

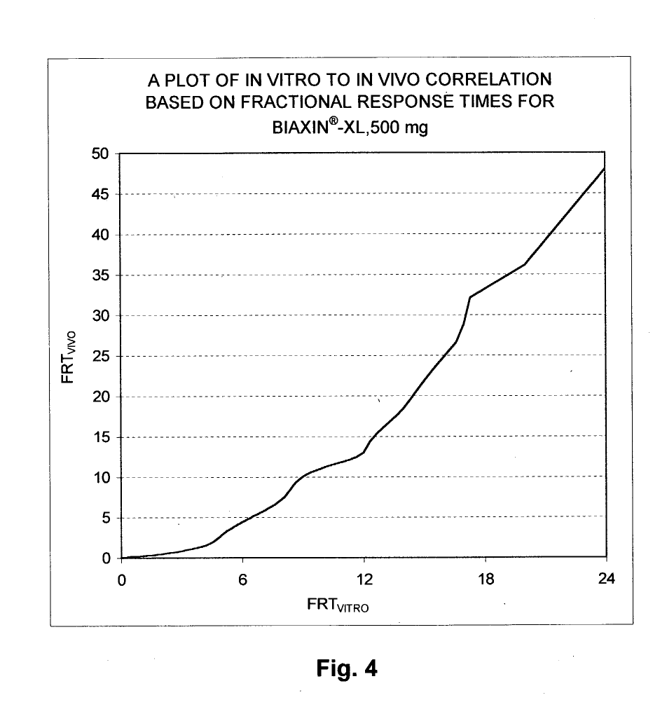

### DETAILED DESCRIPTION OF THE INVENTION

The present invention is a multicomponent system comprising a macrolide, β-cyclodextrin and a dicarboxylic acid to provide a rate-controlled pharmaceutical composition wherein the macrolide is released and absorbed effectively as the delivery system transits along the gastrointestinal tract. The macrolides are poorly soluble and are selected from the group consisting of clarithromycin, dirithromycin, kitasamycin, roxithromycin, rokitamycin, rosaramycin and azithromycin, preferably clarithromycin having a solubility of about one part per 1000 parts of water.

The solubility of clarithromycin as a function of pH is ˜10 mg/ml at pH 2.4, ˜5.5 mg/ml at a pH of 5.4 and about negligible at a pH of 7.4 (when tested in water without a buffer). The presence of a dicarboxylic acid, such as fumaric acid, creates a microenvironment of low pH thereby enhancing the solubility of clarithromycin as it is propelled towards the large intestine from the stomach (10 mg/ml) to the duodenum (5 mg/ml) to the colon (about insoluble at a pH of 7.5 to 8.5).

Within a series of polymer grades of hydroxyl propyl methyl cellulose (Methocel ® series A, E, F and K) which are on the FDA generally recognized as safe list of materials of pharmaceutical necessity, the gel structure is determined by pouring (into a plastic tray) 30 ml of 1% w/v dispersion of Methocel® E4MPCaR, F50 and F4M, respectively, for casting by drying in an air hood at room temperature for over 48 hours. The dried films are exposed to a pH 5.0 as a suitable dissolution medium for Biaxin XL, as reported in USP XIII.

Accordingly, clarithromycin is incorporated into a matrix comprising a hydrophilic polymer, such as hydroxylethylcellulose, hydroxylpropylcellulose, hydroxylpropylmethylcellulose and unmicronized β-cyclodextrin. An appropriate blend is prepared in a planetary mixer by adding MS dispersions to the powder blend. The blend is processed through an extruder consisting of a twin screw system which discharges a wet plastic material through axially positioned screens. Actual extrusion is performed by an extruding roll which forces the damp material through the screens. The feed screws and extruding rollers are chain driven through a variable speed drive (20-85 RPM). The extruded material is dried on a tray in a hot air oven or a vacuum oven. The dried material is milled using an appropriate milling equipment, blended with a lubricant in a dry blender with intermeshing motion for a predetermined time and compressed into a core using appropriately sized tooling equipment on a rotary compression machine. The hydrophilic polymers suitable for extrusion granulation include hydroxypropylmethylcellulose (Metholcel® E and F) are preferred as a result of their ability to be extruded without adversely affecting the extruder.

A major advantage of the present invention is the fact that extrusion granulation material flows uniformly and is compressed into a hard, non friable matrix. These properties of hardness and strength are not accomplished with conventional methods of manufacture because the addition of a buffering agent, such as fumaric acid, to the hydrophilic polymer weakens the structure of the compact material. Another advantage of the composition of the present invention is its virtual pH independence so that variability within and between subjects is minimized in terms of bioperformance.

An example of the present invention is as follows:

### EXAMPLE

A microfluidized multicomponent system of the following components:

| Ingredients | mg/ml | Amount |
|---|---|---|
| Clarithromycin | 500 | 500 |
| β-cyclodextrin | 33 | 33 |
| Fumaric Acid | 100 | 100 |
| | 633 | 633 |
| Hydoxypropylcellulose | 127 | 126 |
| Water (qs 40% w HPC) | 1240.5 | 1240.5 |

Clarithromycin (micronized, d90 ˜30-50μ), β-cyclodextrin (micronized d90˜50μ) and fumaric acid (micronized d90 ˜50μ) are dispersed in a dispersion of the HPC in ware (7% w/w). A small amount of simethicone emulsion is added to defoam the dispersion prior to microfluidization. A blend of a non ionic polymer (150 mg.) and unmicronized β-cyclodextrin (67 mg d90 ˜200μ) with MMS (dry basis) 760 mg. is prepared in a planetary mixer to with is added the Clarithromycin dispersion and is thereafter passed as a damp mass through an extruder. The extruded material is dried on paper lined trays at 45° C. The dried exudates are milled using, respectively, 0.375″ band and 0.063″ band. The sized granulation is lubricated using a blender and then compressed on a rotary tablet machine using round tooling.

The in-vivo dissolution accomplished, based on information available on Biaxin® XL in PDR 2021, converted to approximate single dose for immediate release, is comparable to the XL product.

While the present invention has been described in connection with an exemplary embodiment thereof, it will be understood that many modifications will become apparent to those of ordinary skill in the art; and that this application is intended to cover any adaptations or variations thereof. Therefore, it is manifestly intended that this invention be only limited by the claims and the equivalents thereof.

What is claimed:

1. A one a day release formulation of a macrolide, which comprises:
a) a multicomponent system comprising said macrolide, β-cyclodextrin as a solubility enhancer and, optionally, a minor quantity of a dicarboxylic acid;
b) a non ionic polymer; and
c) a major portion of β-cyclodextrin as an excipient.

2. The one a day release formulation as defined in claim 1 wherein the macrolide is clarithromycin.

3. The one a day release formulation as defined in claim 2 wherein the clarithromycin and the β-cyclodextrin are micronized.

4. The one a day release formulation as defined in claim 3 wherein the dicarboxylic acid is fumaric acid.

5. The one a day release formulation as defined in claim 1 wherein the non ionic polymer is selected from the group consisting of hydroxymethylcellulose, hydroxylpropylcellulose, hydroxylpropylmethylcellulose, polyoxyethylene homopolymer and mixtures thereof.

6. The one a day release formulation as defined in claim 1 wherein said dicarboxylic acid is selected from the group consisting of fumaric acid, succinic acid, malic acid, adipic acid and mixtures thereof.
